(19) United States
(12) Patent Application Publication
(10) Pub. No.: US 2026/0210934 A1
(43) Pub. Date: Jul. 23, 2026

(54) METHOD FOR DETERMINING A MECHANICAL PROPERTY OF AN AGRICULTURAL SOIL

(71) Applicant: COMPAGNIE GENERALE DES ETABLISSEMENTS MICHELIN, Clermont-Ferrand (FR)

(72) Inventors: ANTOINE PATURLE, Clermont-Ferrand (FR); JEREMY VAYSSETTES, Clermont-Ferrand (FR); FRANCOIS PINET, Clermont-Ferrand (FR); MICKAEL LEFEBVRE, Entraigues (FR); ANNIE DUPARQUE, Estrees Mons (FR); DAMIAN MARTIN, Estrees Mons (FR); VINCENT TOMIS, Baralle (FR); OLIVIER SCHEURER, Nyons (FR); CAROLINA UGARTE, Beauvais (FR)

(21) Appl. No.: 18/720,076

(22) PCT Filed: Dec. 13, 2022
(86) PCT No.: PCT/EP2022/085658

§ 371 Date: Jun. 14, 2024

(30) Foreign Application Priority Data
Dec. 16, 2021 (FR) 2113652

(51) Int. Cl. G01N 33/24; G01N 19/00

(52) U.S. Cl. CPC G01N 33/245; G01N 19/00

(57) ABSTRACT

A method for determining the mechanical property of a ground on which a tire mounted on a vehicle is running, the tire being fitted with a sensor configured to acquire a measurement signal representative of the change in the curvature of the tire as it runs over a ground, comprises the following steps: acquiring a measurement signal representative of the change in the curvature of the tire while it is running; determining measurement data comprising (a) a first parameter (KSᵢₙ) representative of a rate at which the tire flattens on contact with the ground, and (b) a second parameter (KSₒᵤₜ) representative of a rate at which the tire regains its shape on becoming separated from the ground; and determining the mechanical property of the ground as a function of the first parameter (KSᵢₙ) and/or the second parameter (KSₒᵤₜ).

### FIELD OF THE INVENTION

The present invention relates to the determination of the mechanical properties of a ground, in particular its spatial evolution in real time, such as the surface deformability. More specifically, the invention proposes determining a condition of mechanical resistance of the ground close to the surface and how it changes after an agricultural implement has been run over it, by means of a measurement signal representative of the circumferential curvature of the tyre.

### TECHNOLOGICAL BACKGROUND

It has proven useful specifically to know, at all times, the mechanical properties of a ground in order to interact with the driver or with the driving assistance systems, so as to inform them in real time of the change in running conditions and possibly react to them. Notably, knowing a condition of mechanical resistance of the ground makes it possible to adjust the use conditions of a vehicle. For example, in the presence of loose ground, the inflation pressure of a tyre may be lowered in order to widen the area of the contact patch in which the tyre is in contact with the ground, and thus to limit the compaction of the ground and the appearance of a rut after the agricultural implement has been run over it.

Furthermore, the local determination of the mechanical resistance of the ground makes it possible to evaluate the influence of the tyre on the mechanical properties of the ground and identify opportunities to carry out, or not carry out, certain operations that depend on this condition of mechanical resistance. For example, running an implement over soil that is too loose may damage the ground or cause the implement to bog down. Working of the soil can also be adversely affected by the condition of mechanical resistance of the ground, such as surface compaction thereof.

By coupling the conditions of mechanical resistance with synchronous geolocation data, it is possible to establish a map of the mechanical resistance of the ground of a plot of land, possibly coupled with other characteristics of the ground. Such a map can prove useful in determining how to improve the ground in the plot of land, for example by installing ground drainage or by modulated loosening depending on the requirements of the ground.

Lastly, the ground, which in particular is agricultural ground, is complex and changes depending on the observation depth. It is characterized by physical variables such as its moisture, its texture and its structure. In general, the moisture of a ground, or its water condition, is defined by the relative mass of water remaining in the ground once its “surface” portion has run off. This is referred to as moisture content by mass or gross weight, expressed as a percentage and designated H. However, the amount of water actually available to a plant, or capillary water, is less than the moisture content by mass since the plant must apply a suction force to take in this water, this being referred to as water potential, expressed in bar. Conventionally, the water potential of a plant is limited to 16 bar, above which the water is not available to the plant. Conversely, a ground at maximum water retention capacity will require a low suction force, of about 0.3 bar, from the plant: the ground is said to be at its “field capacity” of moisture. It is advisable to quantify the capillary water available at a given time or location of the field by calculating the ratio between the measured water content by weight H and its maximum value at the field capacity. HCC refers to this capillary water “normalized” in relation to the field capacity, also expressed as a percentage. Normalizing in relation to the field capacity makes it possible to define a moisture which is independent of the texture of the ground and representative of the water actually available to the plant. The texture of the ground corresponds to the ratio between its constituent clay, loam and sand. These three components are distinguished by their grain size. Thus, a HCC value of 99% means that the capillary water available to the plant corresponds to 99% of the field capacity, which is to say a value very close to the maximum possible value. However, this HCC value of 99% corresponds to a moisture content by weight H of about 40% for a ground with soil of low grain size and high retention of capillary water, such as a clay-based ground, but only to a moisture content by weight H of about 20% for a ground with soil of high grain size and low retention of capillary water, such as a sandy ground.

Lastly, the structure of the ground that corresponds to the three-dimensional arrangement of the material of the ground that it is possible to take note of at various levels, which is to say at different ground depths, is conventionally characterized by its apparent density, designated DA. This parameter can be affected by working of the ground, notably at levels close to the surface, which is to say of several tens of centimetres, but also by the passage of agricultural machines over the field in succession.

The combination of the moisture, the texture and the structure of the ground results in a complex material having specific mechanical properties.

Patent application FR1860481A1 describes a determination of the firmness of the ground in front of the tyre on the sole basis of a measurement of the change in the curvature of a tyre running over said ground. The firmness of the ground is linked to the rate at which the tyre flattens and to the length of the contact patch. The firmness of the ground corresponds to the analysis of the ground over a relatively great depth, this expressing the ability of the ground to bear loads.

However, this method does not make it possible to determine all the physical or mechanical features of the ground that can be of interest for particular applications. In particular, this method does not make it possible to quantify the condition of mechanical resistance of the ground close to the surface, which is to say the analysis of the deformability of the ground over a depth of several tens of centimetres. Similarly, this method also does not make it possible to evaluate the impact the vehicle equipped with the measurement tyre has on the mechanical characteristics of the ground, notably close to the surface, as it runs over the ground.

The aim of the following subjects of the invention is to determine the mechanical properties of the ground and thus evaluate the impact of the tyre running on the ground in real time in order to adapt the running conditions of the tyre as required.

### DESCRIPTION OF THE INVENTION

The invention concerns a method for determining the mechanics of a ground on which a tyre mounted on a vehicle is running, said tyre being fitted with a sensor configured to acquire a measurement signal representative of the change in the curvature of the tyre as it runs over a ground, the method comprising the following steps:
acquiring, by way of the sensor, a measurement signal representative of the change in the curvature of the tyre while it is running,
determining, from the measurement signal, measurement data comprising:
   a) a first parameter representative of a rate, preferably angular rate, at which the tyre flattens on contact with the ground over the course of one revolution of the wheel bearing the tyre, and
   b) a second parameter representative of a rate, preferably angular rate, at which the tyre regains its shape on becoming separated from the ground over the course of one revolution of the wheel bearing the tyre,
determining the mechanical property of the ground as a function of the first parameter and/or the second parameter.

The method makes it possible to easily, precisely and reliably determine the mechanical properties of a ground in real time, preferably at the overall scale of the tyre such as the rut depth but also at a local scale such as the factors of mechanical resistance of the ground over a given depth, from the ground on which a tyre mounted on a vehicle is running, without taking the pressure or load of the vehicle into consideration, from the single measurement signal representative of the change in the curvature of the tyre. Taking the angular rate into account makes it possible to automatically make the running speed of the tyre redundant.

In this instance, the first and second parameters are connected to local effects in the contact patch, which is to say the change in curvature on entering or leaving the contact patch, taking note of the rate of this change. These are not parameters at the overall scale of the contact patch, such as possibly the length of the contact patch. As these variables are local, their sensitivity is much greater than an overall variable at the scale of the contact patch. As a result, taking note of them individually makes it possible to derive the local mechanical properties of the ground on which the tyre is running, this not necessarily being possible, if not to say impossible, with an overall variable, such as the length of the contact patch, which naturally will average the effect over all of the contact patch. The utilization of a single variable makes it possible to centre the analysis on a local region of the contact patch, which is to say in this case the region located at the entry into or exit from the contact patch. By contrast, combining the local variables makes it possible to estimate mechanical properties at the scale of the contact patch by differentiating the mechanical responses from the sensor on entering and on leaving the contact patch.

With preference, the determination of the mechanical property of the ground takes into account its texture, the water condition of the ground, and the depth in order to refine the quality of evaluation of the mechanical property of the ground.

This method is advantageously supplemented by the following features, considered alone or in any technically feasible combination thereof:
during running, over the course of one revolution of the wheel, the curvature of the tyre changes according to a cycle exhibiting:
   a part where there is no contact with the ground,
   a part where there is contact with the ground, wherein the first parameter is determined from part of the measurement signal corresponding to a transition in the curvature of the tyre between the part where there is no contact with the ground and the part where there is contact with the ground, and the second parameter is determined from part of the measurement signal corresponding to a transition in the curvature of the tyre between the part where there is contact with the ground and the part where there is no contact with the ground,
during running, over the course of one revolution of the wheel, the curvature of the tyre changes according to a cycle exhibiting:
   a part where there is no contact with the ground, characterized in the measurement signal by a stable curvature,
   a part where there is contact with the ground, characterized in the measurement signal by a contact curvature variation spike,
   a transition referred to as the coming-into-contact transition between the part where there is no contact with the ground and the part where there is contact with the ground, characterized in the measurement signal by a coming-into-contact curvature variation spike that is the opposite of the contact curvature variation spike,
a transition referred to as the coming-out-of-contact transition between the part where there is contact with the ground and the part where there is no contact with the ground, characterized in the measurement signal by a coming-out-of-contact curvature variation spike that is the opposite of the contact curvature variation spike, the first parameter being determined by a gradient between the coming-into-contact curvature variation spike and the contact curvature variation spike, the second parameter being determined by a gradient between the coming-into-contact curvature variation spike and the contact curvature variation spike,
the mechanical property of the ground is determined using a linear relationship connecting said mechanical property of the ground and the first parameter and/or the second parameter,
the linear relationship takes the following form:

$$F = a + b \times {KS}_{i or j} , or$$

$$F = a + b \times {KS}_{i} + c \times {KS}_{j}$$

where F is a mechanical property factor, KS_{i or j} is the first or the second parameter, and a, b and c are predetermined fixed coefficients,
the mechanical property of the ground is determined by calculating a mechanical property factor from the first parameter and/or the second parameter, and by comparing said mechanical property factor to thresholds delimiting mechanical property categories for the ground,
the mechanical property of the ground is included in the group comprising the rut depth, the mechanical resistance of the ground on entering the contact patch, the mechanical resistance of the ground on leaving the contact patch, and the compaction of the ground caused by the tyre,
the mechanical property of the ground is defined over a ground depth X of less than 40 centimetres, potentially less than 20 centimetres, or even less than 10 centimetres,
a step of locating the vehicle during the step of acquiring the signal that provides an at least two-dimensional position of the vehicle.

In this instance, the rut depth and the compaction of the ground caused by the tyre running over it are considered to be overall variables of the ground at the scale of the tyre. On the other hand, the mechanical resistance of the ground on entering the contact patch and the mechanical resistance of the ground on leaving the contact patch, which correspond to the deformability of the ground close to the surface, are local variables of the ground at the scale of the tyre. The invention also relates to a map of the non-uniformity of the mechanical property of the ground of a surface on which a tyre mounted on a vehicle is running, said tyre being provided with a sensor configured to acquire a measurement signal representative of the change in the curvature of the tyre as it runs over the surface, the method comprising determining the mechanical property of the ground at least twice, corresponding to two at least two-dimensional positions of the vehicle that were obtained by the method according to the invention.

The invention also relates to a tyre comprising a sensor sensitive to the change in the curvature of the tyre and configured to generate a measurement signal representative of the change in the curvature of the tyre as it runs over a ground, comprising an active part and an electronic circuit board, the active part being configured to generate the measurement signal, the electronic circuit board being configured to determine measurement data comprising:
a) a first parameter representative of a rate, preferably angular rate, at which the tyre flattens on contact with the ground over the course of one revolution of the wheel bearing the tyre, and
b) a second parameter representative of a rate, preferably angular rate, at which the tyre regains its shape on becoming separated from the ground over the course of one revolution of the wheel bearing the tyre, the sensor being configured to transmit the measurement data to outside the tyre.

The invention also relates to a data processing unit configured to receive measurement data derived from a measurement signal representative of the change in the curvature of the tyre as it runs over a ground, said measurement data comprising:
a) a first parameter representative of a rate, preferably angular rate, at which the tyre flattens on contact with the ground over the course of one revolution of the wheel bearing the tyre, and
b) a second parameter representative of a rate, preferably angular rate, at which the tyre regains its shape on becoming separated from the ground over the course of one revolution of the wheel bearing the tyre, the data processing unit being configured to determine the mechanical property of the ground as a function of the first parameter and/or the second parameter.

With preference, the data processing unit is configured to receive that at least two-dimensional position of the vehicle that is associated with the measurement data.

The invention also relates to a vehicle comprising:
at least one tyre,
at least one sensor sensitive to the change in the curvature of the tyre and configured to generate a measurement signal representative of the change in the curvature of the tyre as it runs over a ground, and the sensor is preferably disposed inside the tyre,
a data processing unit configured to receive measurement data derived from the measurement signal representative of the change in the curvature of the tyre as it runs over a ground and to determine the mechanical property of the ground as a function of at least one of the measurement data, the measurement data comprising:
   a) a first parameter representative of a rate, preferably angular rate, at which the tyre flattens on contact with the ground over the course of one revolution of the wheel bearing the tyre, and
   b) a second parameter representative of a rate, preferably angular rate, at which the tyre regains its shape on becoming separated from the ground over the course of one revolution of the wheel bearing the tyre, the vehicle being configured to implement the method according to the invention.

The sensor preferably comprises an active part and an electronic circuit board, the active part being configured to generate the measurement signal, the electronic circuit board being configured to determine the measurement data, and wherein the data processing unit is disposed outside the tyre.

The invention also relates to a computer program product comprising program code instructions for executing the method according to the invention when said program is executed on a computer. The computer program product may take the form of a non-transient computer-readable medium that stores code instructions for executing the method according to the invention, when said non-transient computer-readable medium is read by a computer.

### BRIEF DESCRIPTION OF THE DRAWINGS

The invention will be better understood on reading the following description, which is given solely by way of non-limiting example and with reference to the appended figures, in which the same reference numbers denote identical parts throughout, and in which:

FIG. 1 schematically illustrates a tyre mounted on a rim of a vehicle;

FIG. 2 shows an example of a measurement signal read by a sensor sensitive to the curvature of the tyre as the tyre runs;

FIG. 3 shows a flowchart of the steps of the method for evaluating the mechanical property of a ground according to possible embodiments of the invention;

FIG. 4a and FIG. 4b each show an example of the statistical categorization and relationship between the two parameters derived from the measurement signal for a front tyre of a vehicle in various corresponding conditions of local mechanical properties of the ground: the mechanical resistances of the ground on entering and leaving the contact patch;

FIG. 5 shows an example of a map of the non-uniformity of an overall mechanical property of the ground, i.e. the compaction caused by the tyre running over it, on two plots of land, the map being obtained from the measurement signal for one vehicle tyre;

FIG. 6 shows an example of the relationship between the overall mechanical property of the ground, i.e. the rut depth, and one of the parameters derived from the measurement signal for a front tyre of a vehicle.

### DETAILED DESCRIPTION OF EMBODIMENTS

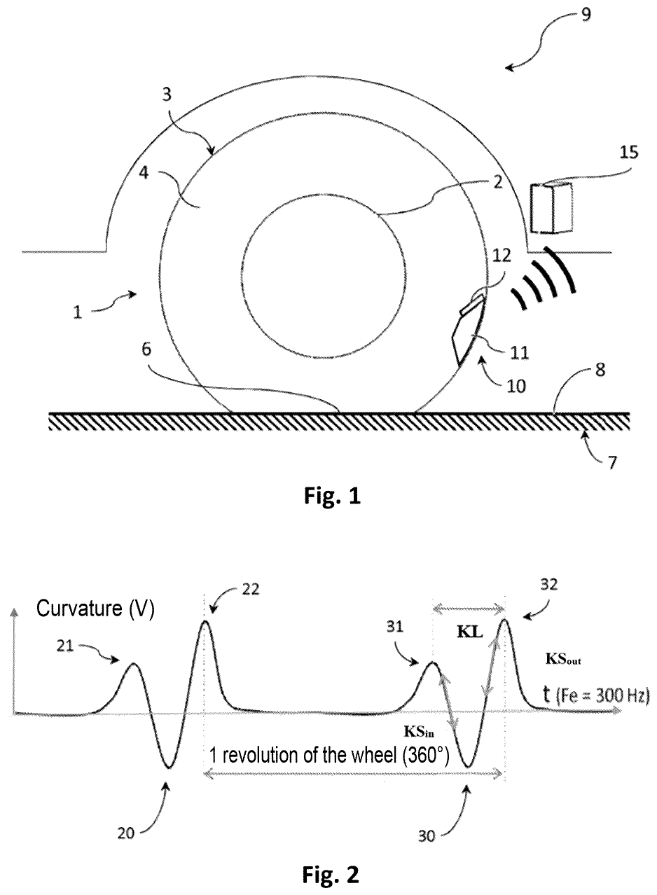

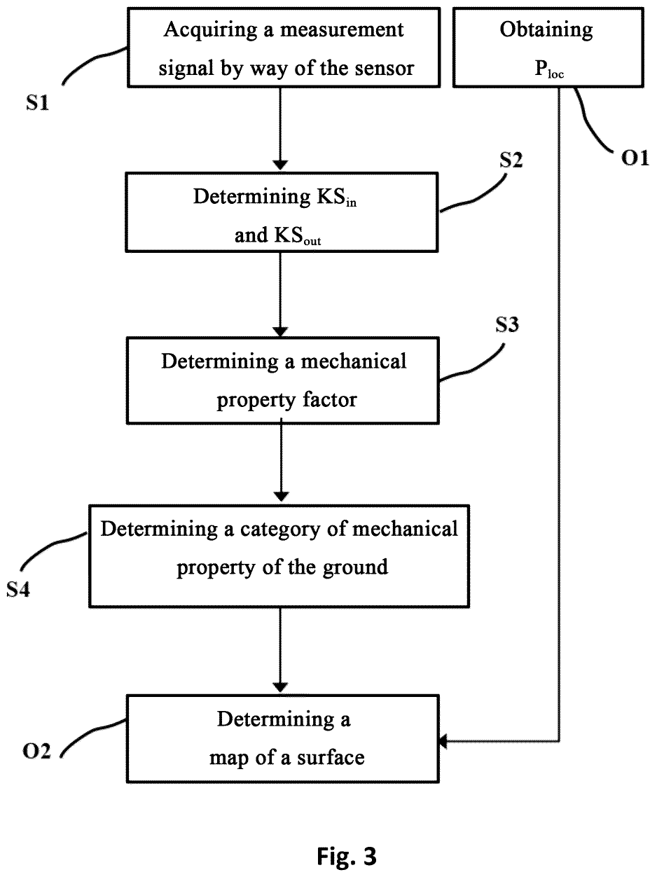

FIG. 1 illustrates a tyre 1 mounted on a rim 2. Such a tyre 1 comprises, on the one hand, a crown region 3, constituting a tread exhibiting tread patterns, and, on the other hand, sidewalls 4 ending in lower sidewall regions. The latter generally comprise a bead wire and a bead for mounting the tyre 1 on the rim 2. The rim 2 is itself connected to the vehicle 9 by an axle (not depicted). The tyre 1 thus forms the contact system providing the connection between the vehicle 9 and the ground 7.

Thus, what is meant by a tyre is a resilient solid designed to be mounted on the rim 2 of a wheel, generally in the form of a tyre band, to form the contact system providing the connection between the vehicle 9 and the ground 7, comprising a tread that undergoes a change in its circumferential radius of curvature when subjected to a load. The tyre 1 is typically made of elastomers (for example rubber) and possibly other textile and/or metallic materials. The tyre 1 may be airless, for example having flexible polyurethane spokes supporting the tread. However, as a preference, a tyre 1 comprises a flexible casing containing a pressurized gaseous interior, typically air. As this is the commonest form of tyre 1, the following description is given non-limitingly with reference to such a tyre 1 having an internal pressure of pressurized gas.

The tyre 1 is subjected to a force applied by the vehicle 9 via the axle and the rim 2 towards the ground 7. This force stems from the axle load, resulting from the weight of the vehicle 9. Because the rim 2 is non-deformable, this force, applied to the tyre 1, deforms the latter when the tyre 1 is in contact with the surface 8 of the ground 7: the part of the crown 3 below the rim 2 flattens, increasing the area of the contact patch 6 in which the tyre 1 is in contact with the ground, while the sidewalls 4 become distended. This deformation is all the more pronounced when the pressure inside the tyre is low and/or when the load carried by the tyre is high. The nature of the ground 7 also has an influence on this deformation, and particularly the mechanical condition of this ground 7. Specifically, resistant ground deforms little if at all, whereas soft or loose ground deforms under the action of the tyre 1, so that the deformation of the tyre 1 is less as part of it is transferred to the ground 7.

The deformation of the tyre 1 results in a change to the circumferential curvature of the tyre 1, namely the curvature of the crown region 3. When the tyre 1 is running, this change to the curvature travels around the circumference of the tyre 1. For a given point on the tyre 1, the curvature will therefore vary periodically with each revolution of the wheel.

The tyre 1 is fitted with a sensor 10 configured to acquire a measurement signal representative of the change in the curvature of the tyre. This sensor 10 is situated inside the casing of the tyre 1. The sensor 10 is preferably situated against the crown region 3. The sensor 10 may be embedded in the structure of the casing of the tyre 1, or may be attached thereto, and for example held in place by an adhesive layer. The sensor 10 comprises an active part 11 secured to the casing of the tyre 1, so that the deformation of the tyre 1 leads to a corresponding deformation of the active part 11 of the sensor 10, which generates a measurement signal that is a function of the deformation of its active part 11. The measurement signal is therefore truly representative of the change in the curvature of the tyre.

As a preference, the sensor 10 is a piezoelectric sensor, which generates a voltage proportional to the variation in bending. More specifically, the sensor 10 may for example comprise an active part 11 made up of a piezoelectric layer between two conducting layers. It is also possible for the sensor 10 to be a resistive sensor, the impedance of which is proportional to the bending of the active part 11 of the sensor. It is also possible to use an accelerometer, although these are far more complex to use and require more processing of the signal. The sensor 10 may also be suited to measuring other parameters, particularly the pressure. The sensor 10 may be incorporated into another piece of electronic equipment installed inside the tyre 1, such as a pressure and/or temperature sensor of TMS (tyre monitoring system) type.

The sensor 10 also comprises an electronic circuit board 12 connected to the active part 11 of the sensor 10 and configured to receive the measurement signal coming from the active part 11. This electronic circuit board 12 comprises at least a processor and a memory, and is suited to processing data such as the measurement signal, in order to determine measurement data from the measurement signal, and to communicate these measurement data. As a preference, the sensor 10 is associated with a wireless transmitter, notably of the radiofrequency type, for example of the type using Bluetooth Low Energy technology, or of the low-power device type operating in the 433 MHz band (LPD 433) able to relay the measurement signal to an automated data processing unit, preferably disposed outside the tyre 1, in order to be processed. The wireless transmitter may form part of the sensor 10, for example as a component of the electronic circuit board 12, or may be separate from the sensor 10. It is thus possible, for example, to provide an antenna inside the tyre 1. In the case of wireless communication, an external receiver may receive the signals sent by the wireless communication means associated with the sensor 10, and relay them to the automated data processing unit.

Of course, the sensor 10 may comprise other elements involved in its correct operation, notably an electrical power supply module, for example consisting of a battery.

When the tyre 1 is running over the ground, the sensor 10 acquires (step S1) the measurement signal representative of the change in the circumferential curvature of the tyre. This measurement signal may be directly connected to the curvature (and therefore be a curvature measurement signal), and therefore monitor the change therein, or may be indirectly connected to the curvature. This is notably the case for a sensor 10 of which the active part 11 is a piezoelectric sensor, because the measurement signal then corresponds to the variation in the curvature which, when integrated, by a charge amplifier for example, becomes the curvature. It is this type of sensor that will be used in the examples which follow. The measurement signal, generated by the active part 11 of the sensor 10, is then processed by the electronic circuit board 12 to determine measurement data from the measurement signal. The purpose of processing the measurement signal is to extract the useful information in this signal, which information is then exploited later on in the method.

FIG. 2 shows a schematic example of a measurement signal read by a sensor 10 sensitive to the curvature of the tyre when the tyre 1 is running. The measurement signal is represented here by its voltage (in V) and designated by curvature, as a function of the rotation of the wheel expressed in degrees.

During running, over the course of one revolution of the wheel, the curvature of the tyre changes according to a cycle exhibiting:
a part where there is no contact with the ground,
a part where there is contact with the ground.

The sequence illustrates two passes into the contact patch in which the tyre 1 is in contact with the ground, of that region in which the sensor 10 is located, which are separated by a part of the cycle where there is no contact with the ground. The part of the cycle where there is no contact with the ground is characterized by a stable curvature, which manifests itself in stability of the measurement signal. The part of the cycle where there is contact with the ground is characterized in the measurement signal by a contact curvature variation spike 20, 30. In FIG. 2, the contact curvature variation spikes 20, 30 are directed downwards. This is because the contact curvature variation spikes 20, 30 correspond to the flattening of the tyre 1 in the contact patch 6, which is to say a curvature close to zero.

The curvature also exhibits a transition referred to as the coming-into-contact transition between the part where there is no contact with the ground and the part where there is contact with the ground, characterized in the measurement signal by a coming-into-contact curvature variation spike 21, 31 that is the opposite of the contact curvature variation spike 20, 30, namely in this instance directed upwards. The variation in curvature also exhibits a transition referred to as the coming-out-of-contact transition between the part where there is contact with the ground and the part where there is no contact with the ground, characterized in the measurement signal by a coming-out-of-contact curvature variation spike 22, 32 that is the opposite of the contact curvature variation spike, namely in this instance directed upwards. The coming-into-contact curvature variation spike 21, 31 and the coming-out-of-contact curvature variation spike 22, 32 correspond to the more or less sudden variations in the radius of curvature of the tyre 1 on entering and leaving the contact patch.

Because the tyre is turning, this same cycle is repeated, with a measurement signal that is stable where there is no contact with the ground, followed by a coming-into-contact curvature variation spike 21, 31, a contact curvature variation spike 20, 30, a coming-out-of-contact curvature variation spike 22, 32, and lastly another stable measurement signal where there is no contact with the ground. This cycle corresponds to one revolution of the wheel, and therefore to 360°, which is shown in FIG. 2. For each cycle, the coming-out-of-contact curvature variation spike 22, 32 affords the major advantage of being a sharp spike and especially of being essentially independent of the conditions of the ground and of the tyre 1. Specifically, the coming-out-of-contact curvature variation spike 22, 32 corresponds to the change in curvature of the tyre 1 on leaving the contact patch, when the region of the tyre 1 in which the sensor 10 is located changes abruptly from the flat state characteristic of the part where there is contact with the ground to the curved state characteristic of the part where there is no contact with the ground. On loose ground, as the tyre 1 runs it compacts the ground beneath it, forming a rut, and therefore a rut bottom that is fairly firm and on which the tyre 1 rests as it leaves the contact patch. Furthermore, the forward progress of the vehicle 9 takes the strain essentially towards entering the contact patch. The tyre 1 on leaving the contact patch thus has a coming-out-of-contact behaviour, in terms of curvature, very similar to the way in which a tyre 1 behaves on a road.

It is thus easy to identify each cycle corresponding to a revolution of the wheel by identifying each coming-out-of-contact curvature variation spike 22, 32. It is also possible to identify the cycles using a dedicated device, such as a rev counter. On that basis, the data can be expressed as a function of the degree of angle in each cycle. That notably means that the cycles and their data can be compared independently of the speed of the vehicle 9. The method steps require just one cycle in order to be implemented, and can therefore be implemented on each cycle. However, in order to make the method more robust with respect to potential isolated unpredictable incidents (the presence of a stone for example), it is possible to use a combination of several measured cycles, for example using a moving average.

The mechanical condition of the ground influences the characteristics of the profile of the measurement signal. The invention therefore seeks to extract parameters from the measurement signal in order to deduce the mechanical condition of the ground therefrom. The method thus comprises determining (step S2), from the measurement signal, measurement data comprising at least one first parameter KSᵢₙ representative of an angular rate at which the tyre flattens on contact with the ground over the course of one revolution of the wheel bearing the tyre 1, and one second parameter KSₒᵤₜ representative of an angular rate at which the tyre regains its shape on becoming separated from the ground over the course of one revolution of the wheel bearing the tyre 1. The measurement data may comprise other parameters or values derived from the measurement signal.

The first parameter KSᵢₙ and the second parameter KSₒᵤₜ are determined from a part of the measurement signal corresponding to a transition of the curvature of the tyre between the part where there is no contact with the ground and the part where there is contact with the ground. More specifically, the first parameter KSᵢₙ is determined from a gradient between the coming-into-contact curvature variation spike 31 and the contact curvature variation spike 30. More specifically, the first parameter KSᵢₙ may correspond to the maximum variation in the curvature between the coming-into-contact curvature variation spike 31 and the contact curvature variation spike 30, which is to say may correspond to the maximum gradient (in terms of absolute value). In the example, with the measurement signal being expressed in volts V as a function of angular degrees °, the first parameter KSᵢₙ may have the units V/°, which is to say may correspond to the first derivative of the curvature of the tyre 1.

The second parameter KSₒᵤₜ is determined from a gradient between the coming-out-of-contact curvature variation spike 32 and the contact curvature variation spike 30. More specifically, the second parameter KSₒᵤₜ may correspond to the maximum variation in the curvature between the coming-out-of-contact curvature variation spike 32 and the contact curvature variation spike 30, which is to say may correspond to the maximum gradient. In the example, with the measurement signal being expressed in volts V as a function of angular degrees °, the second parameter KSₒᵤₜ may have the units V/°, which is to say may correspond to the first derivative of the curvature of the tyre 1.

The parameters KSᵢₙ and KSₒᵤₜ may be approximated in several ways. For example, the parameters KS may correspond to the maximum (in the sense of absolute value) of the derivative of the measurement signal between the coming-into-contact curvature variation spike 31 or the coming-out-of-contact curvature variation spike 32 and the contact curvature variation spike 30, the derivative being estimated from the difference between two successive (or closely-spaced) measurement points, obviously taking their angular separation into consideration. As this is a falling gradient for KSᵢₙ in the example, the same will hold true for KSₒᵤₜ with a rising gradient, and this maximum in the sense of absolute value corresponds to a minimum of the derivative of the measurement signal between the coming-into-contact curvature variation spike 31 and the contact curvature variation spike 30. It is also possible, rather than looking for a derivative extremum, to choose fixed measurement points, such as those situated equidistantly from the peaks of the coming-into-contact curvature variation spike 31 and of the contact curvature variation spike 30, and calculate the derivative using these points. It is also possible to use the measurement points corresponding to a measurement signal value, such as the signal passing through “zero” in the case illustrated. It is also possible to use more complex approaches, such as the Savitzky-Golay algorithm. However, choosing a relatively low sampling frequency, typically less than or equal to 500 Hz, and preferably less than or equal to 400 Hz, such as the frequency of 300 Hz in the example, amounts to smoothing the measurement signal and makes it possible to select approaches, such as those set forth above, that are less demanding in terms of computation.

The first parameter KSᵢₙ and the second parameter KSₒᵤₜ have the advantage of exhibiting a wide degree of variability as a function of the mechanical resistance of the ground, and of being easily obtained, as demonstrated above. More specifically, when the resistance of the ground decreases, the parameters KSᵢₙ and KSₒᵤₜ decrease (in terms of absolute value) and vice versa. As a result, the looser the ground is, the more the rate at which the tyre flattens or the rate at which the tyre regains its shape decreases. Conversely, when the resistance of the ground 7 increases, the parameters KSᵢₙ and KSₒᵤₜ, which is to say the rates of deformation of the tyre, increase.

Considered individually, the parameters KS may depend on the load, the pressure and/or the speed. However, taking into account both the first parameter KSᵢₙ and the second parameter KSₒᵤₜ makes it possible to determine deformation variables of the ground at the scale of the tyre, such as the compaction over a depth X quite close to the surface of the ground C_{tyre} 0-X caused by the passage of the tyre 1, from these same parameters, without knowing the load, the pressure and the speed of the tyre 1 over the ground.

As a preference, it is the electronic circuit board 12 of the sensor 10 which, from the measurement signal, determines the measurement data comprising the first parameter KSᵢₙ and the second parameter KSₒᵤₜ. These measurement data are then transmitted by the sensor 10 to a data processing unit 15 which implements the next part of the method. This data processing unit 15 is preferably disposed outside the tyre 1, for example in the vehicle 9, but the processing unit 15 may also be remote from the vehicle 9, and the transmission of the data may then involve intermediate transmission means. The transmission of the measurement data between the sensor 10 and the data processing unit 15 is then performed wirelessly. The data processing unit 15 typically comprises a processor and a memory and is suited to receiving and processing the measurement data when implementing the next part of the method for determining the mechanical properties of the ground.

It is possible to transmit the measurement signal to the processing unit 15 for implementing the next part of the method. However, determining the measurement data using the sensor 10 and transmitting these measurement data alone to the data processing unit 15 offers the advantage of reducing the amount of data transmitted between the sensor 10 and the data processing unit 15. As the transmission of data uses a great deal of energy, transmitting the measurement data rather than the measurement signal makes it possible to limit the electrical power consumption of the sensor 10, which has limited powering options inside the tyre 1.

It is also advantageous to not use the electronic circuit board 12 of the sensor 10 to implement the next part of the method but rather to use the data processing unit 15 to process the measurement data. This limits the calculations performed by the electronic circuit board 12 of the sensor 10, making it possible to save on power and memory for the electronic circuit board 12. In addition, it is easier to modify the ways in which the next part of the method is implemented on a readily accessible data processing unit 15 rather than on the sensor 10 which is inside the tyre 1.

Once the processing unit 15 has received the measurement data, the processing unit 15 can determine the mechanical property of the ground as a function of the first parameter KSᵢₙ and of the second parameter KSᵢₙ which are present in the measurement data and vary as a function of the mechanical resistance close to the surface of the ground, as shown below.

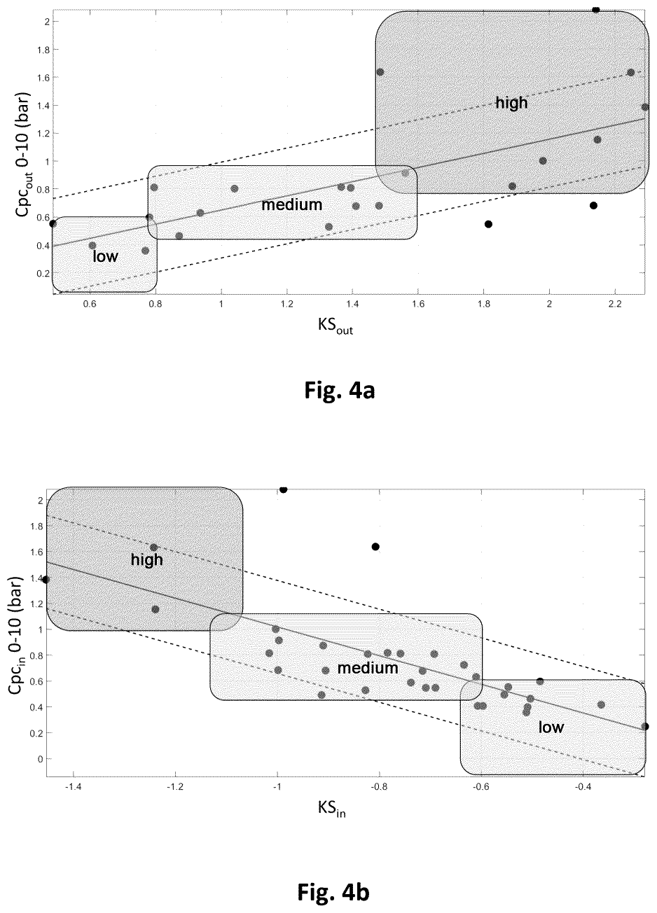

FIGS. 4a and 4b are illustrations of the determination of a mechanical property of the ground which is local and not overall at the scale of the tyre.

In these examples, the measurement data are derived from a measurement signal acquired by a piezoelectric sensor 10 disposed in a front tyre of an offset trailer pulled by an agricultural tractor while said tractor is passing over a ground. This makes it possible to place measuring instruments on the trailer that are necessary to determine the pressure and the load applied to the tyre in stable fashion. The offset of the trailer, in particular of the measurement tyre in relation to the tyres of the tractor, ensures that the ground is not packed down by the passage of the tractor but indeed only by the passage of the measurement tyre.

In the results shown in the two figures FIG. 4a and FIG. 4b, the tractor has run on several types of grounds representing three different texture conditions:
a ground of the medium-loam type,
a ground of the clay-loam type,
a ground of the sandy type.

In addition, these different grounds were placed in all possible water conditions by varying the proportion of water both on the surface and at depth, either naturally depending on the seasons (winter, spring, summer, autumn), or by irrigation.

These grounds were also prepared according to three different initial structural conditions:
A packed-down or compacted condition, designated “W0”, corresponds to the condition of the ground left after a harvest, as a result of which the ground is packed down by an inflated and loaded tyre passing over it many times without any working of the ground after these passages.
A disturbed or soft ground condition made by harrowing the packed-down ground over a depth of 10 or 30 centimetres, designated “W10” and “W30”, respectively.

In addition, other structural conditions of the ground were created by changing the number of times (up to three) the tractor passed over the ground in each of these initial conditions. Lastly, measurements were taken under different load and pressure conditions applied to the tyre that are representative of the use of an agricultural tyre in a field. The measurements were taken for various running speeds below a maximum speed of 20 km/h.

The first mechanical property is the resistance of the ground in front of the tyre, designated Cpcᵢₙ, which corresponds to the determination of the elastic strain limit admissible by the ground expressed in bar, which is to say the maximum strain that the ground can withstand before irreversibly deforming, which will result in a modification of its structure. Necessarily, the lower this strain is, the less resistant or looser the ground is. Conversely, the higher this strain is, the more resistant the ground is. The maximum resistance is obtained for a ground of the road type, which does not deform under the passage of an agricultural tyre in a normal condition of use.

FIG. 4a shows a linear relationship between Cpcᵢₙ and the rate at which the tyre flattens on contact with the ground KSᵢₙ, irrespective of the physical condition of the ground, which is to say its texture, its structure and its water condition. The two straight dashed lines represent the 90% confidence bands.

It is possible to define categories of resistance of the ground particularly in three different levels, thereby making it possible to distinguish the type of resistance of the ground using a laboratory-type characterization of the resistance of the ground obtained by a uniaxial compression measurement on a cylindrical sample of soil taken over a certain depth X. In the case of FIG. 4a, Cpc was measured over a depth of 10 centimetres on core samples of the ground that were taken before or after the passage of the tyre.

By way of non-limiting illustration, the following categories can be used:

TABLE 1

| Cpc (bar) | <=0.5 bar | 0.5-1.0 bar | >1.0 bar |
|---|---|---|---|
| Resistance of the ground | Low or | Medium or | High or |
| | « loose » | « intermediate » | « resistant » |
| KSᵢₙ | >=−0.6 | −1.1-−0.6 | <−1.1 |

A level of correlation was identified between this laboratory measurement Cpc and a measurement of the rate at which the tyre flattens KSᵢₙ, which was obtained using the measurement from the sensor 10 mounted on the tyre in particular for a ground depth of 10 centimetres. It was thus easy to determine the category of resistance of the ground over a depth of 10 centimetres close to the surface before the tyre passes over it solely by measuring the parameter KSᵢₙ, irrespective of the texture, the structure and the water condition of the ground and the conditions of use of the tyre, i.e. the inflation pressure, the load applied and the running speed.

Thus, using F to denote the ground mechanical property factor associated with the resistance of the ground in front of the tyre designated Cpcᵢₙ, and f to denote a function corresponding to the relationship and concerning the parameter KSᵢₙ, it is possible to write the following:

$$F = f ( {KS}_{in} ) \qquad [ Math 1 ]$$

More specifically, the linear relationship may take the following form:

$$F = a + b * {KS}_{in} \qquad [ Math 2 ]$$

where F is the mechanical resistance factor of the ground in front of the tyre over a depth of X centimetres, KSᵢₙ is the rate at which the tyre flattens, and a and b are predetermined non-zero fixed real coefficients.

The fixed coefficients a and b are preferably chosen to maximize the discrimination between mechanical property categories of the ground. It is possible for example to use a one-dimensional discriminant analysis. This discriminant analysis seeks to maximize the separations between the centres of gravity of each of the mechanical property categories of the ground, while at the same time minimizing the spread within the category.

By way of non-limiting illustration, the mechanical resistance factor of the ground in front of the tyre over a depth of 10 centimetres can be determined as follows:

$${Cps}_{in} 0 - 10 ( bar ) = - 0.092 - 1.109 \times {KS}_{in} \qquad [ Math 3 ]$$

The dashed lines around the continuous line that illustrates the above formula delimit the 90% confidence band.

The illustration in FIG. 4b concerns a similar analysis, which was performed on the same grounds in terms of structure, texture and water condition as those of FIG. 4a.

The second mechanical property of which note was taken is the resistance of the ground behind the tyre Cpcₒᵤₜ which corresponds to the determination of the elastic strain limit admissible by the ground, expressed in bar, after the passage of the tyre. If the soil remained elastic, no variation in the resistance of the ground in front of or behind the tyre is observed, which is to say there is no formation of a rut overall. In general, on an agricultural ground, the ground can be seen to be packed down by the passage of the tyre, and it is sought to minimize this by modifying, for example, the inflation pressure of the agricultural tyre. As a result, Cpcₒᵤₜ is generally higher than Cpcᵢₙ, and this expresses the packed-down nature or plastic deformation to which the ground is subject owing to the mechanical action exerted on the ground by the tyre.

It is possible to define categories of resistance of the ground particularly in three different levels, thereby making it possible to distinguish the type of resistance of the ground using a laboratory-type characterization of the resistance of the ground obtained by a uniaxial compression measurement on a cylindrical sample of soil taken over a certain depth X. The same measurements of Cpc as for FIG. 4a were utilized.

By way of non-limiting illustration, the following categories can be used:

TABLE 2

| Cpc (bar) | <=0.5 bar | 0.5-1.0 bar | >1.0 bar |
|---|---|---|---|
| Resistance of the ground | Low or | Medium or | High or |
| | « loose » | « intermediate » | « resistant » |
| KSₒᵤₜ | <=0.8 | 0.8-1.6 | >1.6, |

A similar level of correlation was identified between the laboratory uniaxial compression measurement Cpc, in particular for a ground depth of 10 centimetres, and the determination of the rate at which the tyre regains its shape KSₒᵤₜ, as illustrated in FIG. 4b.

It was thus easy to determine the category of resistance of the ground Cpcₒᵤₜ over a depth of 10 centimetres close to the surface before the tyre passes over it solely by measuring the parameter KSₒᵤₜ, irrespective of the texture, the structure and the water condition of the ground and the conditions of use of the tyre, i.e. the inflation pressure, the load applied and the running speed.

Thus, using F to denote the ground mechanical property factor associated with the resistance of the ground behind the tyre designated Cpcₒᵤₜ, and f to denote a function corresponding to the relationship and concerning the parameter KSₒᵤₜ, it is possible to write the following:

$$F = f ( {KS}_{out} ) \qquad [ Math 4 ]$$

More specifically, the linear relationship may take the following form:

$$F = a + b * {KS}_{out} \qquad [ Math 5 ]$$

where F is the mechanical resistance factor of the ground behind the tyre, KSₒᵤₜ is the rate at which the tyre regains its shape, and a and b are predetermined non-zero fixed real coefficients.

The fixed coefficients a and b are preferably chosen to maximize the discrimination between mechanical property categories of the ground. It is possible for example to use a one-dimensional discriminant analysis. This discriminant analysis seeks to maximize the separations between the centres of gravity of each of the mechanical property categories of the ground, while at the same time minimizing the spread within the category.

By way of non-limiting illustration, the mechanical resistance factor of the ground behind the tyre over a depth of 10 centimetres can be determined as follows:

$${Cps}_{out} 0 - 10 ( bar ) = 0.142 + 0.507 \times {KS}_{out} \qquad [ Math 6 ]$$

The dashed lines around the continuous line that illustrates the above formula delimit the 90% confidence band.

The values for the rate at which the tyre flattens or regains its shape for the same category of ground are different on account of the interaction between the ground and the tyre being different whether entering or leaving the contact patch, thereby justifying the specific relationships for each variable.

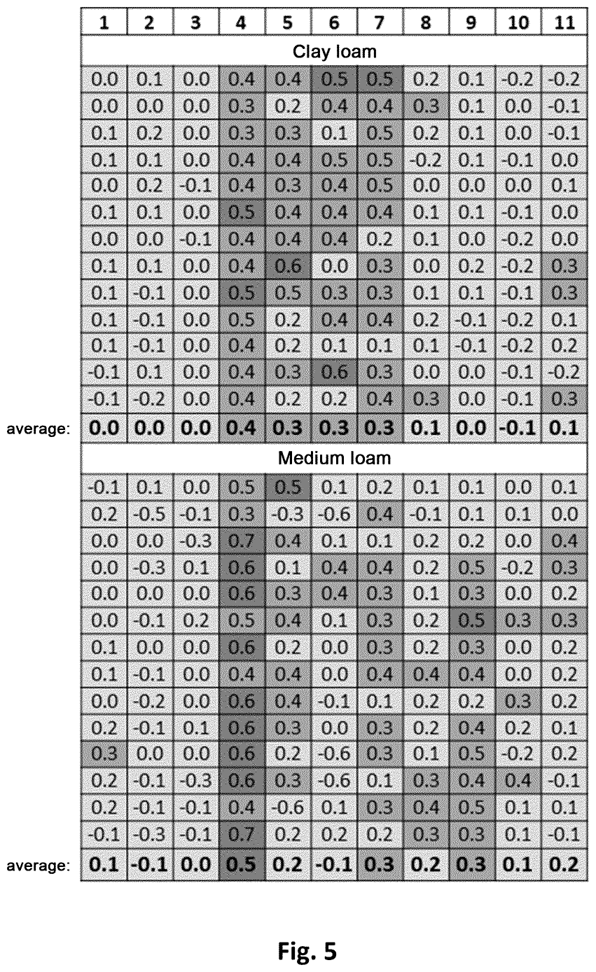

FIG. 5 is a map of the compaction of the ground close to the surface C_{tyre} caused by the passage of the tyre at the scale of two agricultural plots of land distinguished by a different texture but both having a high moisture content through the entire depth of the ground (winter condition).

The compaction of the ground close to the surface C_{tyre} caused by the passage of the tyre is evaluated by comparing the mechanical resistance of the ground in front of the tyre, i.e. Cpcᵢₙ, and behind the tyre, i.e. Cpcₒᵤₜ. In our case, the comparison consists in calculating the difference between the mechanical resistance of the ground after the passage of the tyre and the mechanical resistance of the ground before the passage of the tyre. The compaction is then expressed in bar. However, the comparison may also involve the ratio between the two levels of mechanical resistance of the ground and the compaction is then expressed as a percentage.

The columns of the map represent the longitudinal furrows made by the passage of the tyre equipped with the measuring sensor 10 over the agricultural plot of land under a consistent running condition of the tyre. However, from one furrow to the next, the conditions of use of the tyre can change with an increase in the load applied to the tyre or with a modification of the inflation pressure of the tyre, for example. In the example given, in this case all the plot of land is considered to have the same physical properties in terms of texture and initial structure. However, some furrows have been subjected to specific preparation work that potentially makes the structure of this furrow different from the other furrows. Some furrows in the same plot of land have a structure of the W0, or W10 or W30 type, and a tractor has passed over them up to three times.

For the same furrow, which is to say the same column for a given plot of land, the various lines correspond to linear units of the furrow corresponding to one revolution of the wheel bearing the measurement tyre, i.e. a distance of approximately 5 metres. For each revolution of the wheel, the rates at which the tyre flattens KSᵢₙ and regains its shape KSₒᵤₜ are measured. A Cpcᵢₙ and a Cpcₒᵤₜ are deduced from these values on the basis of the above formulae. The compaction of the ground close to the surface C_{tyre} is then evaluated for each revolution of the wheel. Lastly, the set of values for compaction of the ground close to the surface of each linear unit of the furrow is averaged over the set of linear units of the same furrow.

It is observed that some furrows do not exhibit any compaction of the ground close to the surface, such as in the first columns on the left, this being expressed in the fact that the passage of the tyre does not change the structure of the ground. That is to say, the ground remains in an elastic state following the passage of the tyre in conditions of use of the tyre on these furrows.

On the other hand, other furrows corresponding to furrows 4 to 7 from the left exhibit a high rate of compaction of the ground caused by the tyre, of about 0.3 bars overall, which expresses a high degree of plastic deformation of the ground caused by the passage of the tyre and leads to the formation of a rut. To avoid the formation of a rut, it is then necessary to adapt the conditions of use of the tyre by reducing its inflation pressure or reducing the load it transports. However, if the ground is loose in this furrow, and this will be detected by the relationship linking Cpcᵢₙ to the rate at which the tyre flattens, this packing-down can be desirable. It is necessary to adapt the behaviour depending on the function desired compared to the function obtained.

Such a map shows clear agricultural potential by analysing the potential non-uniformity of the ground of an agricultural plot of land, thereby making it possible to adapt the conditions of use of the tyre a priori to each region of the plot of land in question. In addition, the taking of the measurements and the measurement of the map in real time make it possible to adapt, also in real time, the conditions of use of the tyre while working the plot of land so as to minimize the impact of the agricultural implement on the mechanical property of the ground. For example, these measurements can be used to issue a warning about the risk of formation of a rut detrimental to the exploitation of the plot of land and to adapt the conditions of use of the tyre to the mechanical properties of the ground at the scale of a linear unit of a furrow as quickly as possible.

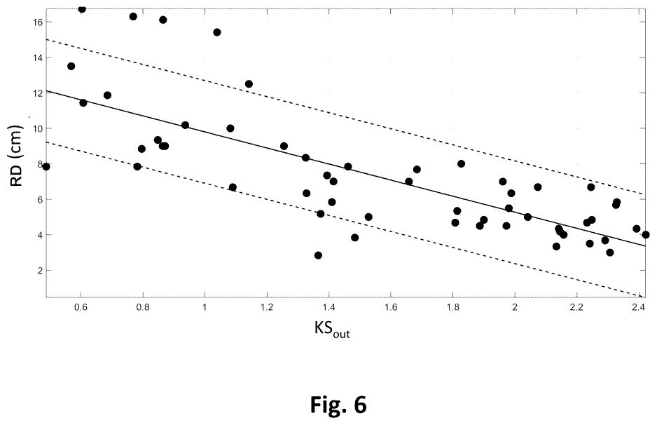

The illustration in FIG. 6 concerns a similar analysis, which was performed on the same grounds in terms of structure, texture and water condition as those of FIG. 4a and FIG. 4b. However, this time around, the mechanical property is overall at the scale of the tyre.

The mechanical property under observation is the rut depth RD which corresponds to the formation of a rut in the agricultural ground, expressed in centimetres, owing to the plastic deformation undergone by the ground after the tyre has passed over it. The fact that it is the resultant of the full passage of the tyre over the ground justifies the overall nature of this mechanical property of the ground. If the soil remained elastic, no plastic deformation of the ground is observed, which is to say there is no formation of a rut. In general, on an agricultural ground of low resistance in nature, the ground can be seen to be packed down by the passage of the tyre, and it is sought to minimize this by modifying, for example, the inflation pressure of the agricultural tyre. As a result, the mechanical action exerted by the tyre on the ground generates a rut of varying depth in the ground.

A level of correlation was identified between the measurement of the rut depth after the passage of the tyre and the rates at which the tyre flattens KSᵢₙ and regains its shape KSₒᵤₜ This is linked to the variation in the plastic limit of the ground as the tyre passes over it, which manifests itself in a variation in the resistances of the ground in front of the tyre, Cpcᵢₙ, and behind the tyre, Cpcₒᵤₜ, over a depth X of the ground.

However, the depth of the ground X to be taken into account in order to form the best correlation with the rut depth depends greatly on the physical properties of the ground.

A level of overall and general correlation was identified between the rut depth RD and the rate at which the tyre regains its shape KSₒᵤₜ by means of a simple relationship between the two variables.

Specifically, the correlation links this terrain measurement, after the passage of the tyre, with a measurement of the rate at which the tyre regains its shape KSₒᵤₜ, which was obtained using the measurement from the sensor 10 mounted on the tyre in particular for a ground depth of 10 centimetres. It was thus easy to determine the rut depth in the ground after the tyre passes over it solely by measuring the parameter KSₒᵤₜ, irrespective of the texture, the structure and the water condition of the ground and the conditions of use of the tyre, i.e. the inflation pressure, the load applied and the running speed.

Thus, using F to denote the ground mechanical property factor associated with the rut depth RD, and f to denote a function corresponding to the relationship and concerning the parameter KSₒᵤₜ, it is possible to write the following:

$$F = f ( {KS}_{out} ) ; or \qquad [ Math 7 a ]$$

$$F = f ( {KS}_{out} , {KS}_{in} ) \qquad [ Math 7 b ]$$

More specifically, the linear relationship may also take the following form:

$$F = a + b * {KS}_{out} ; or \qquad [ Math 8 a ]$$

$$F = a + b * {KS}_{out} + c * {KS}_{in} \qquad [ Math 8 b ]$$

where F is the factor of rut depth, KSₒᵤₜ is the rate at which the tyre regains its shape, KSᵢₙ is the rate at which the tyre flattens, and a, b and c are predetermined non-zero fixed real coefficients.

By way of non-limiting illustration, the factor of rut depth RD can be determined from the rate at which the tyre regains its shape KSₒᵤₜ, as follows:

$$PO ( cm ) = 14.3 - 4.5 \times {KS}_{out} \qquad [ Math 9 ]$$

The dashed lines around the continuous line that illustrates the above formula delimit the 90% confidence band.

1.-14. (canceled)

15. A method for determining a mechanical property of a ground on which a tire mounted on a vehicle is running, the tire being fitted with a sensor configured to acquire a measurement signal representative of a change in a curvature of the tire as it runs over the ground, the method comprising the following steps:
acquiring, using the sensor, a measurement signal representative of the change in the curvature of the tire while it is running;
determining, from the measurement signal, measurement data comprising:
(a) a first parameter KSᵢₙ representative of a rate at which the tire flattens on contact with the ground over a course of one revolution of a wheel bearing the tire, and
(b) a second parameter KSₒᵤₜ representative of a rate at which the tire regains shape on becoming separated from the ground over the course of one revolution of the wheel bearing the tire; and
determining the mechanical property of the ground as a function of the first parameter KSᵢₙ and/or the second parameter KSₒᵤₜ.

16. The method according to claim 15, wherein, during running, over the course of one revolution of the wheel, the curvature of the tire changes according to a cycle exhibiting:
a part where there is no contact with the ground, and
a part where there is contact with the ground, wherein the first parameter KSᵢₙ is determined from a part of the measurement signal corresponding to a transition in the curvature of the tire between the part where there is no contact with the ground and the part where there is contact with the ground, and the second parameter KSₒᵤₜ is determined from a part of the measurement signal corresponding to a transition in the curvature of the tire between the part where there is contact with the ground and the part where there is no contact with the ground.

17. The method according to claim 15, wherein, during running, over the course of one revolution of the wheel, the curvature of the tire changes according to a cycle exhibiting:
a part where there is no contact with the ground, characterized in the measurement signal by a stable curvature,
a part where there is contact with the ground, characterized in the measurement signal by a contact curvature variation spike,
a coming-into-contact transition between the part where there is no contact with the ground and the part where there is contact with the ground, characterized in the measurement signal by a coming-into-contact curvature variation spike that is opposite of the contact curvature variation spike, and
a coming-out-of-contact transition between the part where there is contact with the ground and the part where there is no contact with the ground, characterized in the measurement signal by a coming-out-of-contact curvature variation spike that is opposite of the contact curvature variation spike, the first parameter KSᵢₙ being determined by a gradient between the coming-into-contact curvature variation spike and the contact curvature variation spike, and the second parameter KSₒᵤₜ being determined by a gradient between the coming-into-contact curvature variation spike and the contact curvature variation spike.

18. The method according to claim 15, wherein the mechanical property of the ground is determined using a linear relationship connecting the mechanical property of the ground and the first parameter KSᵢₙ and/or the second parameter KSₒᵤₜ.

19. The method according to claim 18, wherein the linear relationship takes the following form:
$$F = a + b \times K {S}_{i or j} , or$$
$$F = a + b \times K {S}_{i} + c \times K {S}_{j}$$
where F is a mechanical property factor, KS_{i or j} is the first or the second parameter, and a, b and c are predetermined fixed coefficients.

20. The method according to claim 15, wherein the mechanical property of the ground is determined by calculating a mechanical property factor F from the first parameter KSᵢₙ and/or the second parameter KSₒᵤₜ, and by comparing the mechanical property factor to thresholds delimiting mechanical property categories for the ground.

21. The method according to claim 15, wherein the mechanical property of the ground is included in the group comprising a rut depth RD, a mechanical resistance of the ground on entering a contact patch Cpcᵢₙ, a mechanical resistance of the ground on leaving the contact patch Cpcₒᵤₜ, and a compaction of the ground caused by the tire C_{tyre}.

22. The method according to claim 15, wherein the mechanical property of the ground is defined over a ground depth X of less than 40 centimeters.

23. The method according to claim 15, further comprising a step of locating the vehicle during the step of acquiring the signal that provides an at least two-dimensional position Ploc of the vehicle.

24. A tire comprising a sensor sensitive to a change in a curvature of the tire and configured to generate a measurement signal representative of the change in the curvature of the tire as it runs over a ground, comprising an active part and an electronic circuit board, the active part being configured to generate the measurement signal, the electronic circuit board being configured to determine measurement data comprising:
(a) a first parameter KSᵢₙ representative of a rate at which the tire flattens on contact with the ground over a course of one revolution of a wheel bearing the tire, and
(b) a second parameter KSₒᵤₜ representative of a rate at which the tire regains shape on becoming separated from the ground over the course of one revolution of the wheel bearing the tire, the sensor being configured to transmit the measurement data to outside the tire.

25. A data processing unit configured to receive measurement data derived from a measurement signal representative of a change in a curvature of a tire as it runs over a ground, the measurement data comprising:
(a) a first parameter KSᵢₙ representative of a rate at which the tire flattens on contact with the ground over a course of one revolution of a wheel bearing the tire, and
(b) a second parameter KSₒᵤₜ representative of a rate at which the tire regains shape on becoming separated from the ground over the course of one revolution of the wheel bearing the tire,
the data processing unit being configured to determine the mechanical property of the ground as a function of the first parameter KSᵢₙ and/or the second parameter KSₒᵤₜ.

26. A vehicle comprising:
at least one tire;
at least one sensor sensitive to a change in a curvature of the tire and configured to generate a measurement signal representative of the change in the curvature of the tire as it runs over a ground;
a data processing unit configured to receive measurement data derived from the measurement signal representative of the change in the curvature of the tire as it runs over a ground and to determine the mechanical property of the ground as a function of at least one of the measurement data, the measurement data comprising:
(a) a first parameter KSᵢₙ representative of a rate at which the tire flattens on contact with the ground over a course of one revolution of the wheel bearing the tire, and
(b) a second parameter KSₒᵤₜ representative of a rate at which the tire regains shape on becoming separated from the ground over the course of one revolution of the wheel bearing the tire, the vehicle being configured to implement the method according to claim 15.

27. The vehicle according to claim 26, wherein the at least one sensor is disposed inside the tire.

28. The vehicle according to claim 27, wherein the at least one sensor comprises an active part and an electronic circuit board, the active part being configured to generate the measurement signal, the electronic circuit board being configured to determine the measurement data, and
wherein the data processing unit is disposed outside the tire.
